(19) United States
(12) Patent Application Publication
(10) Pub. No.: US 2018/0296130 A1
(43) Pub. Date: Oct. 18, 2018

(54) Device for Taking Anatomical Measurements

(71) Applicant: Dominick Borrelli, Brooklyn, NY (US)

(72) Inventor: Dominick Borrelli, Brooklyn, NY (US)

(21) Appl. No.: 15/816,428

(22) Filed: Nov. 17, 2017

Related U.S. Application Data
(60) Provisional application No. 62/485,133, filed on Apr. 13, 2017.

(51) Int. Cl. A61B 5/107

(52) U.S. Cl. CPC A61B 5/1072

(57) ABSTRACT

A device for taking anatomical measurements uses an L-shaped beam, a clamp and an adjustment handle to measure the dimensions of body parts. The L-shaped beam is a rigid structure that functions as the main arm and the fixed jaw blade of a pair of lockable measurement calipers. Specifically, the L-shaped beam has a first leg and a second leg that are terminally connected to form a right angle. The clamp is a sleeved device that is slidably mounted onto the first leg of the L-shaped beam. This enables the user to slide the clamp along the first leg until the clamp is moved into a desired position. The user can then lock the clamp in place. The adjustment handle is a rigid beam that is terminally connected to the clamp. Thus positioned, the adjustment handle acts as the movable jaw blade for the pair of lockable measurement calipers.

The current application claims a priority to the U.S. Provisional Patent application serial number 62/485,133 filed on Apr. 13, 2017.

### FIELD OF THE INVENTION

The present invention generally relates to a measuring device. More specifically, the present invention is a waist measuring device, that allows users to obtain accurate measurements that can be transferred to garments.

### BACKGROUND OF THE INVENTION

Tailoring is one of the oldest professions in the world. While a large majority of clothes and accessories are, mass produced in factories to fit a wide range of the population, when professional expertise is required, tailors are the most common solution. Tailor services are available locally depending on where the person lives. Custom made apparel, such as suits, and uniforms require the physical presence of a costumer at a tailor shop for accurate individualized measurements. In addition, it is not uncommon for tailors to ask the costumer to come back for various test fittings before the final article of clothing is finalized. Issues arise if the person is not mobile, and requires assistance to obtain measurements. Obtaining accurate measurements without physically being present, requires the use of various tools. A common method is to use a piece of string, and wrap it around the user's waist, and measure the circumference using a tape measure or a straight edge. While this method can provide accurate results, it can be challenging to accomplish and requires the multiple steps to obtain a measurement. Trying on various clothes in stores requires patience. Various sizes of the same garment are required, increasing the time the user spends to accomplish such a simple task. The present invention aims to solve some of the problems mentioned above by allowing users to gather waist measurements in one step.

### BRIEF DESCRIPTION OF THE DRAWINGS

FIG. 1 is an isometric perspective view of the present invention.

FIG. 2 is a front view of the present invention.

FIG. 3 is a rear view of the present invention.

FIG. 4 is a right-side exploded view of the present invention.

FIG. 5 is a front exploded sectional view of the present invention taken along line 5-5 in FIG. 4.

### DETAIL DESCRIPTIONS OF THE INVENTION

All illustrations of the drawings are for the purpose of describing selected versions of the present invention and are not intended to limit the scope of the present invention.

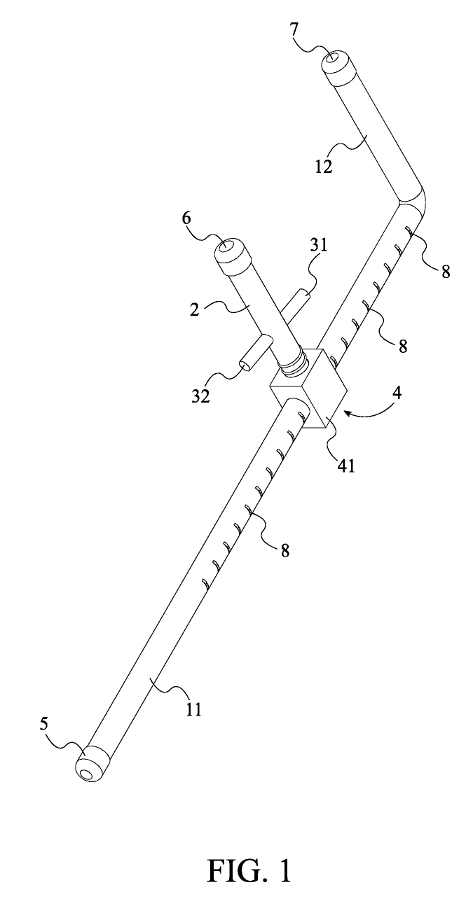

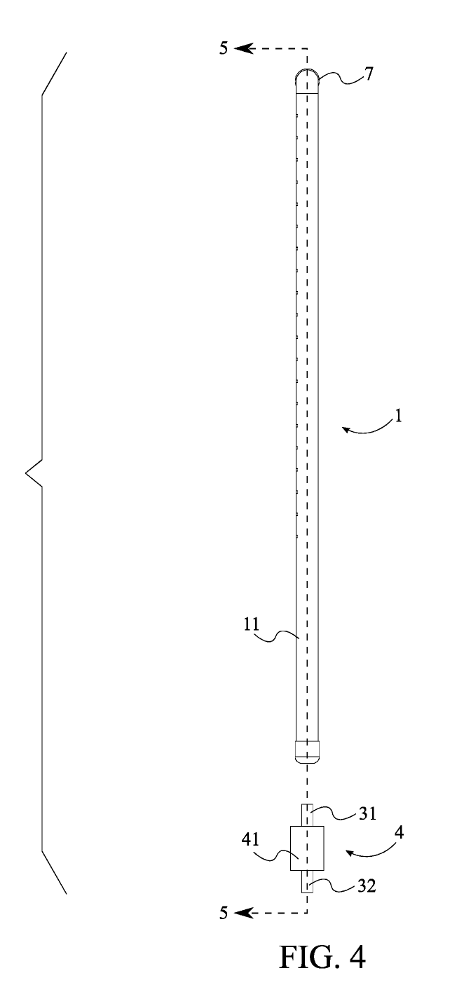

As can be seen in FIG. 1 through FIG. 5, the preferred embodiment of the present invention is a device for taking anatomical measurements. The present invention makes use of a pair of lockable measurement calipers to accurately measure the dimensions of an individual's body parts. Additionally, the present invention is designed to be a handheld device that enables a user the employ a single tool when measuring the dimensions of various body parts. To accomplish this, the present invention comprises an L-shaped beam 1, a clamp 4, and an adjustment handle 2. The L-shaped beam 1 is a rigid structure that is grasped by the user when making measurements. Additionally, the L-shaped beam 1 comprises a first leg 11 and a second leg 12. Furthermore, the first leg 11 is terminally connected to the second leg 12, Finally, the first leg 11 is positioned perpendicular to the second leg 12. As a result, the first leg 11 and the second leg 12 of the L-shaped beam 1 are positioned to act as the main arm and the fixed jaw blade of the pair of lockable measurement calipers. Specifically, the first leg 11 is the main arm that is held in the hands of a tailor, and the second leg 12 is the fixed jaw blade placed against a client's body. The clamp 4 acts as the slide portion of the present invention. Furthermore, the clamp 4 is a lockable carriage device that, while disengaged, is able to freely slide along the length of the first leg 11. Specifically, the clamp 4 is slidably mounted along the first leg 11 so that the clamp 4 can be slid along the length of the first leg 11. The adjustment handle 2 is a protrusion that is grasped by the hand of the user. Additionally, the clamp 4 is terminally connected to the adjustment handle 2. Consequently, the user is able to engage and disengage the clamp 4 by twisting the adjustment handle 2. The user also employs the adjustment handle 2 to reposition the clamp 4 along the length of the first leg 11. Finally, the adjustment handle 2 is positioned parallel to the second leg 12. As a result, the adjustment handle 2 is able to function as the movable jaw blade of the pair of lockable measurement calipers.

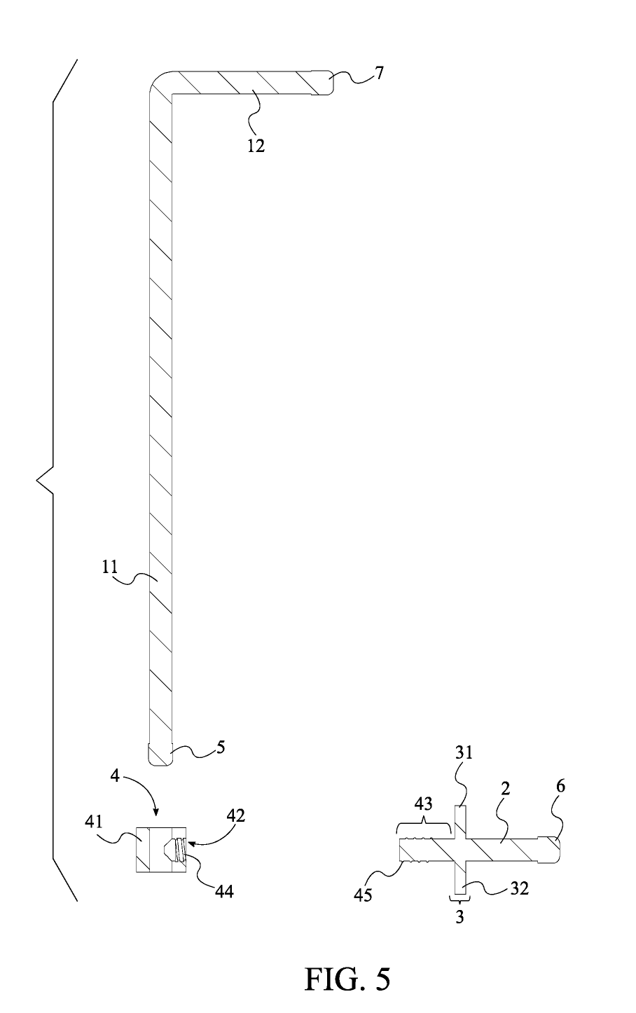

As can be seen in FIG. 1 and FIG. 5, the present invention makes use of a multicomponent clamp 4 that latches onto the first leg 11 and can be locked in place once a measurement has been taken. To accomplish this, the clamp 4 comprises a sleeve 41, a clamp hole 42, and a movable jaw 43. The sleeve 41 is a rigid hollow tube that acts as the fixed jaw of the clamp 4. Additionally, the sleeve 41 is slidably mounted along the first leg 11. As a result, the sleeve 41 is able to move along the length of the first leg 11, until the clamp 4 is engaged. While the sleeve 41 enables the clamp 4 to slide along the first leg 11, the clamp hole 42 and the movable jaw 43 work in concert to enable the clamp 4 to become engaged, and thus locked into place. As such, the clamp hole 42 traverses into the sleeve 41. Additionally, the movable jaw 43 engages into the clamp hole 42. Furthermore, a first end of the movable jaw 43 is pressed against a lateral surface of the first leg 11. As a result, the movable jaw 43 is able to travel through the clamp hole 42 and then press against the first leg 11. Thus, securing the clamp 4 at a desired position along the first leg 11.

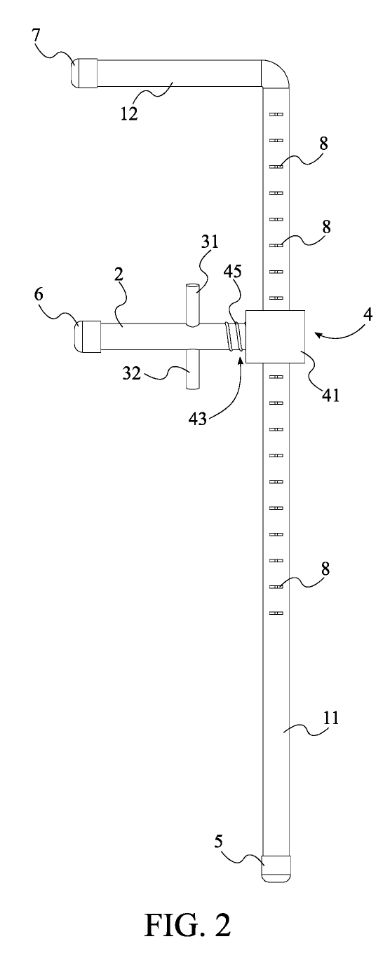

As can be seen in FIG. 2 and FIG. 5, the adjustment handle 2 enables the user to easily reposition the clamp 4. To facilitate this, the adjustment handle 2 is connected adjacent to a second end of the movable jaw 43, opposite to the first leg 11. Additionally, the adjustment handle 2 is colinearly aligned to the movable jaw 43. Accordingly, rotating the adjustment handle 2 enables the user to engage the first end of the movable jaw 43 through the clamp hole 42. The engagement between the first end of the movable jaw 43 and the clamp hole 42 is preferably established by a threaded connection. Specifically, the present invention comprises a female threading 44 and a male threading 45. The female threading 44 is laterally connected within the clamp hole 42. Additionally, the male threading 45 is laterally connected around the first end of the movable jaw 43. Finally, the male threading 45 engages the female threading 44. As a result, the movable jaw 43 can be pressed against the first leg 11 by rotating the adjustment handle 2, such that the male threading 45 engages with the female threading 44.

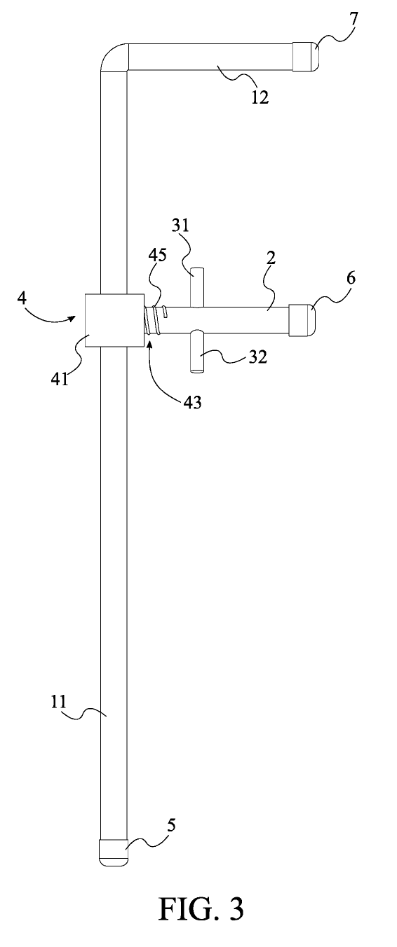

As can be seen in FIG. 3 and FIG. 5, to facilitate engaging the clamp 4, the present invention comprises a handle grip 3. The handle grip 3 is an ergonomically designed attachment that enables the user to more easily manipulate the adjustment handle 2, and thus the movable jaw 43 of the clamp 4. As such, the handle grip 3 is laterally mounted onto the adjustment handle 2. Additionally, the handle grip 3 is positioned adjacent to the terminal connection between the adjustment handle 2 and the clamp 4. Accordingly, the handle grip 3 facilitates grasping the adjustment handle 2. In a first alternative embodiment of the present invention, the handle grip 3 comprises a first rod 31 and a second rod 32. The first rod 31 and the second rod 32 are rigid protrusions that extend from the adjustment handle 2, and facilitate rotating the movable jaw 43. As such, the adjustment handle 2 is terminally connected to the first rod 31. Additionally, the first rod 31 is positioned perpendicular to the adjustment handle 2. Furthermore, the adjustment handle 2 is terminally connected to the second rod 32. Moreover, the second rod 32 is positioned perpendicular to the adjustment handle 2. Finally, the first rod 31 is colinearly aligned to the second rod 32. Consequently, the adjustment handle 2 is positioned in between the first rod 31 and the second rod 32 so that the user can gain increased leverage when rotating the movable jaw 43.

As can be seen in FIG. 1 and FIG. 5, the present invention is designed with several components that facilitate use and prevent damage to the person or piece of clothing being measured. Specifically, the present invention comprises a first endcap 6, a second endcap 7, a stopper 5, and a plurality of measurement indicia 8. The first endcap 6 and the second endcap 7 are rubberized covering devices. As such, the first endcap 6 is terminally connected to the adjustment handle 2, opposite to the clamp 4. Additionally, the second endcap 7 is terminally connected to the second leg 12, opposite to the first leg 11. As a result, the first endcap 6 and the second endcap 7 are positioned between the ends of the adjustment handle 2 and the second leg 12, and the garment or body part being measured. This arrangement of components prevents the present invention from damaging the garment or harming the person being measured. The stopper 5 is an attachment that limits the distance that the clamp 4 is able to travel along the first leg 11. Specifically, the stopper 5 is terminally connected to the first leg 11, opposite to the second leg 12. Accordingly, the stopper 5 prevents the clamp 4 from sliding off of the first leg 11. The plurality of measurement indicia 8 is a graduated scale that is superimposed onto the first leg 11. Furthermore, each of the plurality of indicia is serially distributed along the first leg 11. Consequently, the user is able to take an accurate measurement of a body part by placing the second leg 12 at one side of the body part and then sliding the clamp 4 along the second leg 12. The user engages the clamp 4 once the adjustment handle 2 is moved to the correct place to the body part being measured. The user is then able to ascertain the distance between the second leg 12 and the adjustment handle 2 by visually inspecting the position of the clamp 4 relative to the plurality of measurement indicia 8.

Although the invention has been explained in relation to its preferred embodiment, it is to be understood that many other possible modifications and variations can be made without departing from the spirit and scope of the invention as hereinafter claimed.

1. A device for taking anatomical measurements comprising:
an L-shaped beam;
a clamp;
an adjustment handle module;
the L-shaped beam comprising a first leg and a second leg;
the first leg being terminally connected to the second leg;
the first leg being positioned perpendicular to the second leg;
the clamp being slidably mounted along the first leg;
the clamp being terminally connected to the adjustment handle module;
the adjustment handle module being positioned parallel to the second leg;
the clamp comprising a sleeve, a sleeve hole, a clamp hole and a female threading;
the sleeve hole penetrating the sleeve;
the sleeve hole being parallelly oriented to the first leg;
the first leg being slidably inserted into the sleeve hole;
the clamp hole traversing into the sleeve;
the clamp hole being parallelly oriented to the second leg;
the sleeve hole and the clamp hole being communicated with each other;
the female threading being laterally connected within the clamp hole;
the adjustment handle module comprising an adjustment handle, a movable jaw and a male threading;
the adjustment handle and the movable jaw being fixed to each other;
the movable jaw being located in between the adjustment handle and the sleeve;
the male threading being laterally connected around the movable jaw;
the movable jaw being engaged into the clamp hole by the male threading engaging the female threading; and
the movable jaw being configured to be pressed against the first leg.

2. The device for taking anatomical measurements as claimed in claim 1 comprising:
the adjustment handle being colinearly aligned to the movable jaw.

3. (canceled)

4. The device for taking anatomical measurements as claimed in claim 1 comprising:
a handle grip;
the handle grip being laterally mounted onto the adjustment handle and the movable jaw; and
the handle grip being positioned in between the adjustment handle and the movable jaw.

5. The device for taking anatomical measurements as claimed in claim 4 comprising:
the handle grip comprising a first rod and a second rod;
the first rod being positioned perpendicular to the adjustment handle and the movable jaw;
the second rod being positioned perpendicular to the adjustment handle and the movable jaw; and
the first rod being colinearly aligned to the second rod.

6. The device for taking anatomical measurements as claimed in claim 1 comprising:
a first endcap; and
the first endcap being terminally connected to the adjustment handle, opposite to the movable jaw.

7. The device for taking anatomical measurements as claimed in claim 1 comprising:
a second endcap; and
the second endcap being terminally connected to the second leg, opposite to the first leg.

8. The device for taking anatomical measurements as claimed in claim 1 comprising:
a stopper; and
the stopper being terminally connected to the first leg, opposite to the second leg.

9. The device for taking anatomical measurements as claimed in claim 1 comprising:
a plurality of measurement indicia;
the plurality of measurement indicia being superimposed onto the first leg; and
the plurality of measurement indicia being serially distributed along the first leg.

10. (canceled)

11. (canceled)

12. (canceled)

13. (canceled)

14. (canceled)

15. (canceled)

16. (canceled)
